(19) United States
(12) Patent Application Publication
(10) Pub. No.: US 2025/0389616 A1
(43) Pub. Date: Dec. 25, 2025

(54) TUNABLE SYNTHETIC EYE MODEL

(71) Applicant: ALCON INC., Fribourg (CH)

(72) Inventors: Jing XU, Irvine, CA (US); John PARK, Irvine, CA (US); Yu YAN, Irvine, CA (US)

(21) Appl. No.: 19/238,603

(22) Filed: Jun. 16, 2025

Related U.S. Application Data
(60) Provisional application No. 63/663,208, filed on Jun. 24, 2024.

(51) Int. Cl. G01M 11/02; A61B 3/13

(52) U.S. Cl. CPC G01M 11/0207; A61B 3/13

(57) ABSTRACT

A tunable eye model includes a housing having an inner chamber and at least one aperture adjacent to an anterior portion of the inner chamber. A lens is at least partially disposed in the at least one aperture, and the lens has a variable focal length. An optical target of the tunable eye model is disposed over a posterior portion of the inner chamber. The optical target is configured to facilitate evaluation of a characteristic of an ophthalmic microscope when viewed through the lens with the ophthalmic microscope.

### BACKGROUND

Anatomically, the human eye is divided into two distinct regions: the anterior segment and the posterior segment. The anterior segment includes the lens and extends from the outermost layer of the cornea to the posterior of the lens capsule. The posterior segment of the eye includes the anterior hyaloid membrane and all of the ocular structures behind it, such as the retina, choroid, the optic nerve, and vitreous, which is a gel-like substance that can include “floaters” (clumps/strands of cells that cast shadows on the retina). Vitreoretinal surgery is performed within the posterior segment of the human eye using an ophthalmic microscope in order to treat serious conditions such as retinal detachment.

Notably, the lens is disposed between the ophthalmic microscope and the posterior segment of the human eye. Accordingly, any visualization of the posterior segment is performed through the lens, which adds a complex variable to the optical path for such visualization. For example, the lens can be natural or artificial. Different types of artificial lenses have different optical properties and multiple properties of natural lenses vary from person to person. As a result of this variability, it is challenging to evaluate characteristics of ophthalmic microscopes in a manner that adequately considers all of the scenarios that may be encountered during a surgical procedure.

There have been improvements in the art, such as improved microscope designs and manufacturing processes. However, these improvements have not overcome the challenges associated with visualization through the lens of the human eye. Therefore, improved systems and techniques for evaluating characteristics of ophthalmic microscopes are desirable.

### SUMMARY

Aspects of the present disclosure relate to ophthalmic visualization, and more specifically, to evaluating a characteristic of an ophthalmic microscope.

In certain embodiments, an eye model is provided. The eye model includes a housing having an inner chamber and at least one opening adjacent to an anterior portion of the inner chamber. A lens is at least partially disposed in the at least one opening, and the lens has a variable focal length. An optical target is disposed over a posterior portion of the inner chamber. The optical target is configured to facilitate evaluation of a characteristic of an ophthalmic microscope when viewed through the lens with the ophthalmic microscope.

In certain embodiments, a device includes a housing comprising an inner chamber. A first interchangeable lens is at least partially disposed in the housing. The first interchangeable lens is of a set of interchangeable lenses for evaluating one or more characteristics of an ophthalmic microscope. A curved optical target is disposed over a portion of the inner chamber. The curved optical target is configured to facilitate evaluation of a first characteristic of the ophthalmic microscope when viewed through the first interchangeable lens with the ophthalmic microscope.

In certain embodiments, a method includes orienting an ophthalmic microscope relative to a lens at least partially disposed in an inner chamber of a housing of an eye model. The lens having a first variable focal length. The method also includes evaluating a characteristic of the ophthalmic microscope through the lens having the first variable focal length. The evaluating is based on an optical target disposed over a posterior portion of the inner chamber.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a representation of a human eye, according to embodiments described herein.

FIG. 2 illustrates a representation of a cross-sectional view of a tunable synthetic eye model, according to embodiments described herein.

FIG. 3 illustrates a representation of a cross-sectional view of a tunable synthetic eye model with alternative lenses, according to embodiments described herein.

FIG. 4 illustrates a representation of a cross-sectional view of a tunable synthetic eye model with an integrated light source, according to embodiments described herein.

FIGS. 5A and 5B illustrate a tunable synthetic eye model with an external light source, according to embodiments described herein.

FIG. 6 illustrates a representation of a cross-sectional view of a tunable synthetic eye model with a tunable optical element, according to embodiments described herein.

FIG. 7 illustrates a representation of example optical targets, according to embodiments described herein.

FIG. 8 illustrates a method for evaluating a characteristic of an ophthalmic microscope, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to ophthalmic microscopes, and more specifically, to evaluating a characteristic of an ophthalmic microscope.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical console or a standalone light source.

Note also that, as described herein, an inferior end, segment, or portion of a component refers to the end, segment, or portion that is beneath or lower such as a bottom or underside of a tissue or structure. Conversely, a superior end, segment, or portion of the component refers to the end, segment, or portion that is above or higher such as a top or topside of the tissue or structure.

Further note that, as described herein, a medial end, segment, or portion of a component refers to the end, segment, or portion that is closest to an inside or a midline of a body. On the other hand, a lateral end, segment, or portion of the component refers to the end, segment, or portion that is closest to an outside of the body or furthest from the midline of the body.

Note that, as described herein, an anterior end, segment, or portion of a component refers to the end, segment, or portion that is before or in front such as a front or front side of a tissue or structure. Conversely, a posterior end, segment, or portion of the component refers to the end, segment, or portion that is behind or in back such as a back or backside of the tissue or structure.

As used herein, the term “about” may refer to a +/−10% variation from the nominal value. It is to be understood that such a variation can be included in any value provided herein.

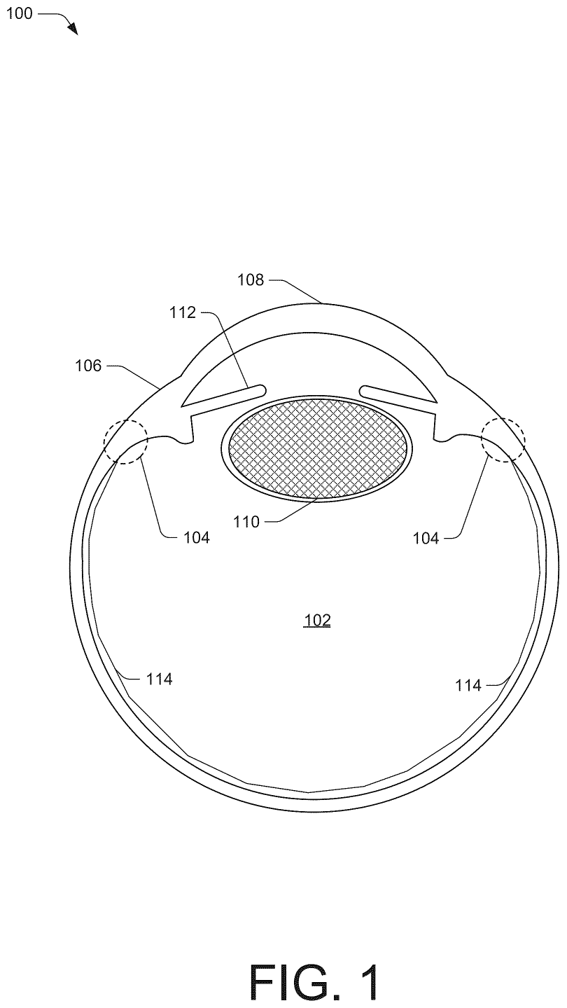

FIG. 1 illustrates a representation of a human eye 100, according to embodiments described herein. As depicted in FIG. 1, the eye 100 illustrates a vitreous chamber 102, a pars plana 104, a sclera 106, a cornea 108, a lens 110, an iris 112, and a retina 114. The vitreous chamber 102 is located in the posterior segment of the eye between the lens 110 and the retina 114. The vitreous chamber 102 is filled with vitreous, which can include a vitreous “floater.”

The pars plana 104 is a region within the ciliary body commonly utilized to access the posterior segment during vitreoretinal surgical procedures (e.g., to remove portions of the vitreous “floater”). This access is typically achieved via cannulas that are inserted into small incisions made in the pars plana 104 during the procedure. For instance, the cannulas may control an intraocular pressure and/or mitigate trauma to ocular tissue from inserting/removing various surgical instruments.

The sclera 106 is the white/opaque fibrous tissue that is the structural layer of the outer eye and forms its round shape. The sclera 106 extends from the cornea 108 (the transparent front surface of the eye) to the optic nerve at the back of the eye. The cornea 108 covers the iris 112, which is the colored part of the eye that controls the size of the pupil. The pupil allows light into the eye, which the lens 110 focuses on the retina 114 at the back of the eye. The retina 114 includes photoreceptor cells that convert the light into signals for visual perception.

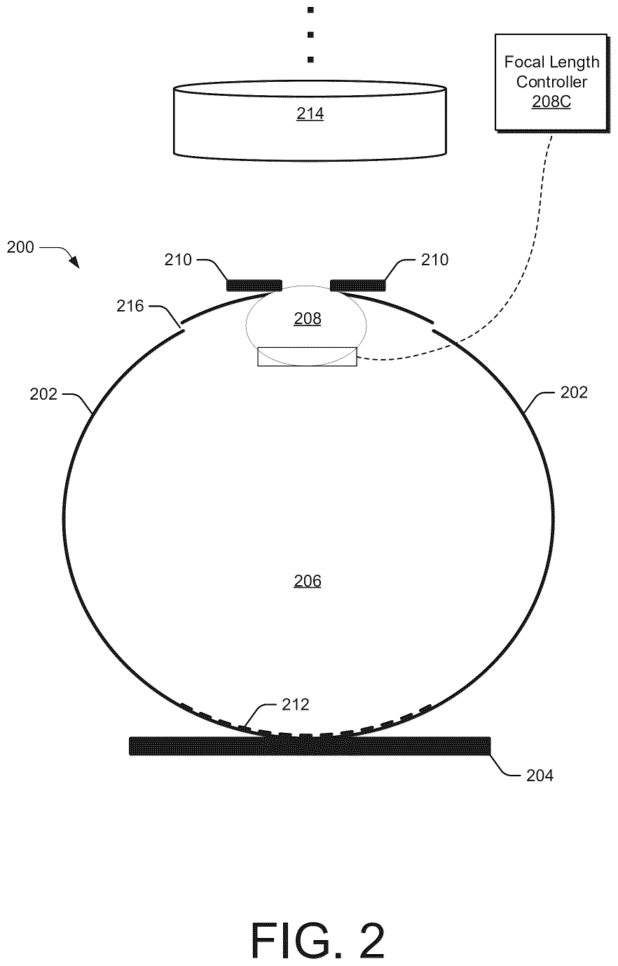

FIG. 2 illustrates a representation of a cross-sectional view of a tunable synthetic eye model 200, according to embodiments described herein. Generally, the eye model 200 is designed to represent the eye 100. As shown, the eye model 200 includes a housing 202 which is illustrated to include a base 204 configured to interface with a flat surface. For instance, the housing 202 has a shape that is generally semi-spherical, and the housing 202 includes an inner chamber 206. In some embodiments, the inner chamber 206 has dimensions configured to simulate (e.g., match) dimensions of the vitreous chamber 102 of the human eye. The housing 202 can be manufactured from a variety of materials such as polymers, metals, ceramics, composites, etc.

In one or more embodiments, the inner chamber 206 includes a material (e.g., a fluid) configured to simulate optical properties within the vitreous chamber 102 of the human eye. In various examples, the inner chamber 206 may be filled with water, a balanced salt solution (BSS), a gel, vitreous, one or more biological materials, etc. In some examples, the inner chamber 206 can comprise a simulated vitreous humor (e.g., having a pH of about 7.4). In one or more examples, the inner chamber 206 comprises a material with optical properties similar or corresponding to (e.g., within at least a +/−10 to 20% range) optical properties of the vitreous humor within the vitreous chamber 102 of the human eye. In the one or more examples, the inner chamber 206 may comprise a material with optical properties similar or corresponding to (e.g., within at least a +/−10 to 20% range) optical properties of BSS. In some embodiments, the inner chamber 206 is filled with a material configured to simulate one or more occurrences that can be encountered during a vitreoretinal surgical procedure. For example, the inner chamber 206 can include a fluid/material at least partially comprising blood or a fluid/material configured to have optical properties of blood in order to simulate hemorrhaging or a traumatic injury. In some embodiments, the inner chamber 206 is at least partially filled with a material configured to have optical properties that simulate optical properties within the vitreous chamber 102 caused by one or more patient conditions such as age, disease (e.g., diabetes), various medications, etc.

A lens 208 is at least partially disposed in an opening 216 of the housing 202 in some embodiments. In various embodiments, the lens 208 may be configured to simulate the natural lens 110 and/or an artificial lens used to replace the natural lens 110 of the human eye. In one or more embodiments, the lens 208 is a focus tunable lens such as a liquid lens with a variable focal length. In examples in which the lens 208 is focus tunable with a variable focal length, a voltage applied to an electroactive material (e.g., an electroactive polymer, a piezoelectric material, an electrowetting material, etc.) of the lens 208 can be varied to change a shape (e.g., a thickness, a curvature, etc.) of the lens 208 and adjust the variable focal length. For instance, adjusting (increasing or decreasing) a voltage applied to the lens 208 causes an adjustment to the variable focal length of the lens 208.

In some embodiments in which the lens 208 is focus tunable with a variable focal length, the eye model 200 may include a focal length controller 208C configured to adjust a thickness, curvature, and focal length of the lens 208. In one or more examples, a user can specify a particular focal length via user interaction with the focal length controller 208C, and the focal length controller 208C applies a particular voltage to the electroactive material of the lens 208. The application of the particular voltage to the electroactive material changes the shape (e.g., the thickness, the curvature, etc.) of the lens 208 to adjust the variable focal length to the particular focal length.

Although in the illustrated example the focal length controller 208C applies the particular voltage to the electroactive material via a wired connection, it is to be appreciated that, in some embodiments, the focal length controller 208C causes the particular voltage to be applied to the electroactive material via a wireless connection. In various embodiments, the lens 208 is configured to simulate multiple conditions of the human eye, e.g., by adjusting the variable focal length of the lens 208 to different particular focal lengths. By adjusting the variable thickness, curvature, and/or refractive index of the lens 208 to adjust the variable focal length of the lens 208, the lens 208 is capable of simulating the natural lens 110 of the human eye with different conditions such as a nearsightedness, a farsightedness, etc.

In some embodiments, the lens 208 has an optical power in a range of −100 to 100 diopters. In other embodiments, the lens 208 has an optical power that is less than −100 diopters or greater than 100 diopters. In one or more examples, the lens 208 can be configured to simulate different amounts of progression from a healthy natural lens 110 to a cataractous lens 110 of the human eye. In some examples, the lens 208 can be configured to simulate a cataractous lens 110 through the use of material with reduced transparency, causing light scattering in the lens 208, increasing light absorption by the lens 208, etc. As such, the lens 208 may be configured to simulate any artificial lens or natural lens 110 encountered during a vitreoretinal surgical procedure, e.g., when viewing the retina 114.

The eye model 200 is illustrated as including an adjustable aperture 210 disposed above, or radially outward from, the lens 208 relative to the housing 202. In some embodiments, the adjustable aperture 210 is configured to simulate the iris 112 by varying an amount of light allowed to pass through the lens 208 and into the inner chamber 206. The adjustable aperture 210 can be mechanically adjusted, electronically adjusted, manually adjusted, and/or the like. In one or more examples, the adjustable aperture 210 operates in a manner similar to a camera aperture or shutter to vary the amount of light allowed to pass through the lens 208 and into the inner chamber 206. For example, adjustable aperture 210 may include a simple leaf-type shutter, a guillotine-type shutter, or a diaphragm-type shutter. By varying the amount of light allowed to pass through the lens 208 and into the inner chamber 206, the adjustable aperture 210 can adjust visibility of an optical target 212 using an ophthalmic microscope 214.

The ophthalmic microscope 214 can generally include an analog microscope or a digital microscope. In one example, the ophthalmic microscope 214 includes a fundus camera. In another example, the ophthalmic microscope 214 includes a slit lamp. Although FIG. 2 depicts a non-contact example of the ophthalmic microscope 214, it is to be appreciated that the ophthalmic microscope 214 is also capable of use in contact examples in some embodiments. In certain embodiments, the ophthalmic microscope 214 may include a contact lens (e.g., a macular lens, a wide-angle ocular lens, etc.) for contacting the cornea 108 after applying gel to the surface of the cornea 108. In embodiments in which the contact lens is utilized with the eye model 200 to evaluate one or more characteristics of the ophthalmic microscope 214, the contact lens may contact a portion of the adjustable aperture 210 and/or the lens 208 with or without applying the gel to the portion of the adjustable aperture 210 and/or the lens 208.

In one or more embodiments, the optical target 212 is disposed over a posterior portion of the inner chamber 206 such that the lens 208 is disposed between the ophthalmic microscope 214 and the optical target 212. In such embodiments, the location of the optical target 212 simulates a location of a portion of the retina 114 in the human eye. In FIG. 2, the optical target 212 is illustrated as being curved like the retina 114; however, in some examples, the optical target 212 may be shaped differently than the retina 114. For example, a portion of the optical target 212 can extend a distance into the inner chamber 206, similar to abnormal blood vessels extending a distance into the vitreous chamber 102 of the human eye.

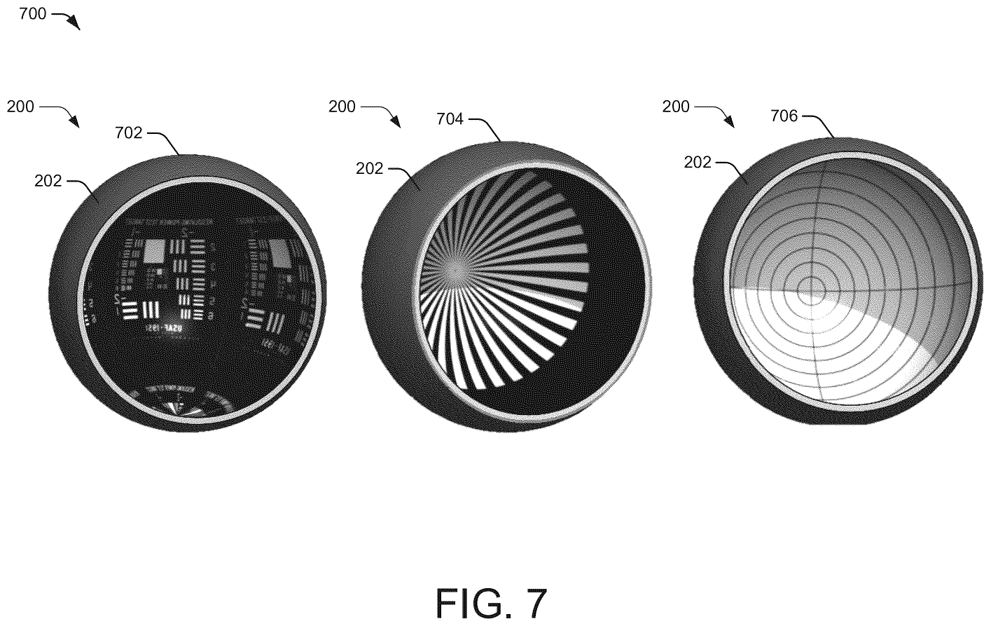

In various examples, the optical target 212 may be fabricated by etching (e.g., chemical or laser etching), additive manufacturing, lithographic processes, printing processes, etc. In some embodiments, the optical target 212 is a high-resolution target that includes multiple reference marks or symbols. As such, based on known dimensions, spacing, and/or other features of the reference marks or symbols, a characteristic of the ophthalmic microscope 214 may be evaluated. Examples of characteristics of the ophthalmic microscope 214 which can be evaluated using the optical target 212 include, but are not limited to, modulation transfer function (MTF), distortion, relative illumination, field of view (FOV), depth of field (DOF), etc. In various embodiments, the optical target 212 can be exchangeable with another optical target 212 configured to evaluate different characteristics of the ophthalmic microscope 214 as described in greater detail with respect to FIG. 7.

Further, using the optical target 212, characteristics of the ophthalmic microscope 214 may be evaluated under a variety of different conditions typically encountered during a vitreoretinal surgical procedure. As an example, vitreoretinal surgical procedures may take place under various lighting conditions, depending on a variety of factors within the operating room. As such, the adjustable aperture 210 can vary (increase or decrease) an amount of light allowed to pass through the lens 208 and into the inner chamber 206 for visualization of the optical target 212 using the ophthalmic microscope 214 (e.g., to simulate operating/examining environments with different lighting conditions). In another example, different patients may have natural lenses with varying visual acuity. As such, a focal length of the lens 208 can be increased or decreased to vary the optical power of the lens 208 (e.g., to simulate lenses of patients with varying degrees of visual acuity).

In another example, patients may have natural lenses in varying conditions, such as healthy, cataractous, etc. As such, the opacity of the lens 208 may be increased (e.g., to simulate cataractous lenses of patients) or decreased (e.g., to simulate healthy/artificial lenses of patients). In certain embodiments, the lens 208 may include layers (e.g., two layers) of liquid crystal, and the opacity of the lens 208 can be changed by changing polarizations of the layers in substantially real time.

Further, the vitreous chamber 102 of a patient's eye may include different material during different stages of a vitreoretinal surgical procedure. For example, the vitreous within the vitreous chamber 102 may be replaced with other material such as BSS. As such, the material included within the inner chamber 206 of the eye model 200 can be varied to simulate BSS, vitreous, and/or the like.

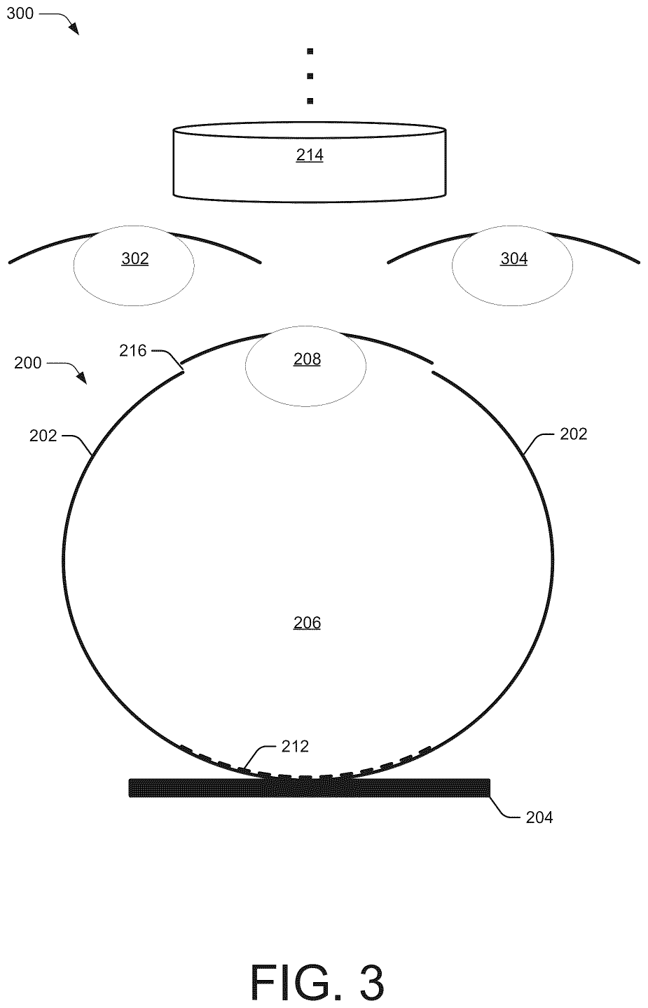

FIG. 3 illustrates a representation 300 of a cross-sectional view of a tunable synthetic eye model 200 with alternative lenses, according to embodiments described herein. For instance, the representation 300 includes the lens 208, as well as alternative lenses 302, 304. In one or more embodiments, the lens 208 and the alternative lenses 302, 304 are part of a system for evaluating performance characteristics of the ophthalmic microscope 214. For example, the alternative lenses 302, 304 may be interchangeably introduced into the representation 300 in place of the lens 208.

In some embodiments, the lens 208 and the alternative lenses 302, 304 each have different optical properties. In one or more examples, the lens 208 has a first optical power, alternative lens 302 has a second optical power, and alternative lens 304 has a third optical power. In various embodiments, the lens 208 is configured to simulate an artificial lens of the human eye; the alternative lens 302 is configured to simulate a healthy natural lens 110 of the human eye; and the alternative lens 304 is configured to simulate a cataractous lens 110 of the human eye. In such various embodiments, the lens 208 and the alternative lenses 302, 304 may each have the same optical power or the lens 208 and the alternative lenses 302, 304 can each have a different optical power. In some examples, the lens 208 is a first type of artificial lens (e.g., manufactured by a first manufacturer), the alternative lens 302 is a second type of artificial lens (e.g., manufactured by a second manufacturer), and the alternative lens 304 is a third type of artificial lens (e.g., manufactured by a third manufacturer).

It is to be appreciated that the lens 208 and the alternative lenses 302, 304 are each usable to evaluate a characteristic of the ophthalmic microscope 214. In some embodiments, the lens 208 and the alternative lenses 302, 304 are each configured to evaluate a different characteristic of the ophthalmic microscope 214. In other embodiments, the lens 208 and the alternative lenses 302, 304 are each usable to evaluate the same characteristic of the ophthalmic microscope 214 (e.g., under different patient or environmental conditions).

For example, a reference marker of the optical target 212 can be visualized with the ophthalmic microscope 214 through the lens 208 to evaluate, e.g., distortion of the ophthalmic microscope 214. In this example, the lens 208 can be replaced with the alternative lens 302, and the reference marker of the optical target 212 may be visualized with the ophthalmic microscope 214 through the alternative lens 302 in order to evaluate the distortion of the ophthalmic microscope 214. In one or more embodiments, the distortion of the ophthalmic microscope 214 when using the lens 208 can be compared to the distortion of the ophthalmic microscope 214 when using the alternative lens 302. Similarly, the alternative lens 302 may be replaced with the alternative lens 304 such that the distortion of the ophthalmic microscope 214 may be evaluated through the alternative lens 304 by visualizing the reference marker of the optical target 212. In various embodiments, the distortion of the ophthalmic microscope 214 when using the lens 208 may be compared to the distortion of the ophthalmic microscope 214 when using the alternative lens 304. Further, the distortion of the ophthalmic microscope 214 when using the alternative lens 302 can also be compared to the distortion of the ophthalmic microscope 214 when using the alternative lens 304.

In some embodiments, the lens 208 and the alternative lenses 302, 304 are included as part of a kit, e.g., for evaluating a performance characteristic of the ophthalmic microscope 214 and another ophthalmic microscope. For instance, the kit may be used to determine whether the ophthalmic microscope 214 and the other ophthalmic microscope are equivalent relative to the performance characteristic. In various examples, the lens 208 and the alternative lenses 302, 304 are used as part of a quality control inspection process such that if a particular glyph or feature of the optical target 212 is visible when the optical target 212 is viewed using the ophthalmic microscope 214 through each of the lens 208 and the alternative lenses 302, 304, then the ophthalmic microscope 214 passes the quality control inspection process.

In one or more examples, the lens 208 and the alternative lens 302 and/or the alternative lens 304 are included in a set of interchangeable lenses for evaluating characteristics of the ophthalmic microscope 214. In various embodiments, the optical target 212 is configured to facilitate evaluation of a first characteristic of the ophthalmic microscope 214 through the lens 208. For instance, the optical target 212 may be configured to facilitate evaluation of a second characteristic of the ophthalmic microscope 214 through the alternative lens 302. In some examples, the first characteristic may be a FOV of the ophthalmic microscope 214 and the second characteristic can be a DOF of the ophthalmic microscope 214. In other examples, the first characteristic may be a distortion of the ophthalmic microscope 214 and the second characteristic can be a relative illumination of the ophthalmic microscope 214. In some embodiments, the optical target 212 is configured to facilitate evaluation of a third characteristic of the ophthalmic microscope 214. For example, the third characteristic can be the ophthalmic microscope's 214 MTF, FOV, DOF, relative illumination, distortion, etc.

Although the alternative lenses 302, 304 are described as alternatives to the lens 208, it is to be appreciated that in one or more embodiments, the alternative lenses 302, 304 can be additional lenses. For example, in order to achieve a particular visual effect, the lens 208 and the alternative lens 302 may be used simultaneously (e.g., in a series). In this example, the lens 208 can be a focus tunable lens with a variable focal length. In another example in which the lens 208 and the alternative lens 302 are used simultaneously, the lens 208 may be a focus tunable lens and the alternative lens 302 may be an additional focus tunable lens. In some examples, the lens 208 may be usable simultaneously with both of the alternative lenses 302, 304 (e.g., in a series).

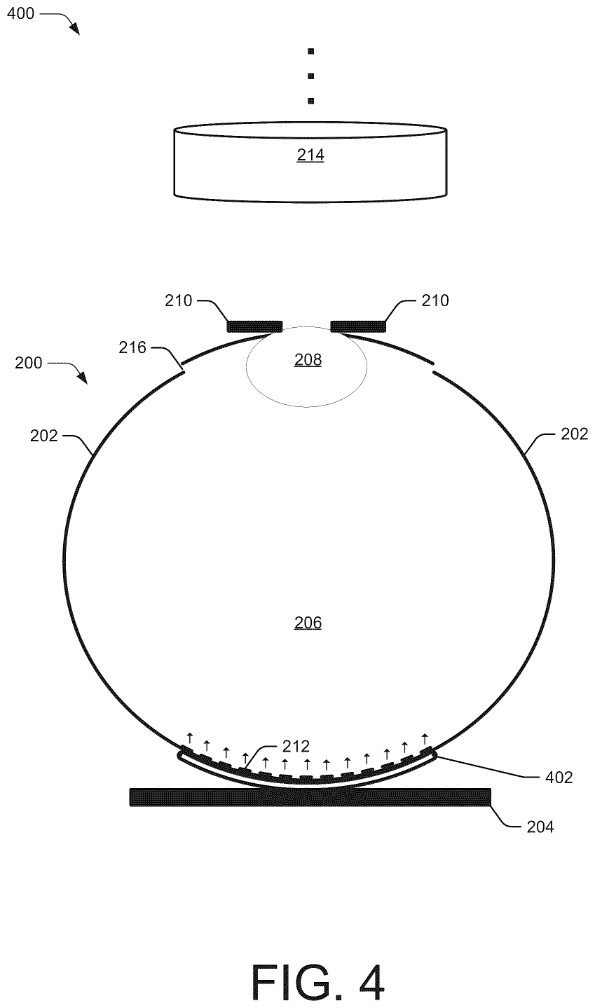

FIG. 4 illustrates a representation 400 of a cross-sectional view of a tunable synthetic eye model 200 with an integrated light source, according to embodiments described herein. As shown, the representation 400 includes a light source 402, which projects light into the inner chamber 206. In some embodiments, the light source 402 includes one or more light emitting diodes configured to illuminate (e.g., back-illuminate) the optical target 212. In one or more embodiments, the light source 402 can be disposed on either or both sides of the optical target 212 to vary directions of light used to illuminate the optical target 212.

In the example illustrated in FIG. 4, the light source 402 is curved and provides uniform illumination of the optical target 212. However, in other examples, the light source 402 may be flat or other shapes. For example, the light source 402 is capable of providing non-uniform illumination of the optical target 212. In various embodiments, the light source 402 is configured to simulate lighting conditions that can be encountered during a vitreoretinal surgical procedure. In one or more examples, the light source 402 may provide no more than a threshold amount of light to illuminate the optical target 212. In certain embodiments, colors (wavelengths) and/or intensities of light emitted from the light source 402 can be varied to simulate the lighting conditions that can be encountered during the vitreoretinal surgical procedure or to simulate other lighting conditions.

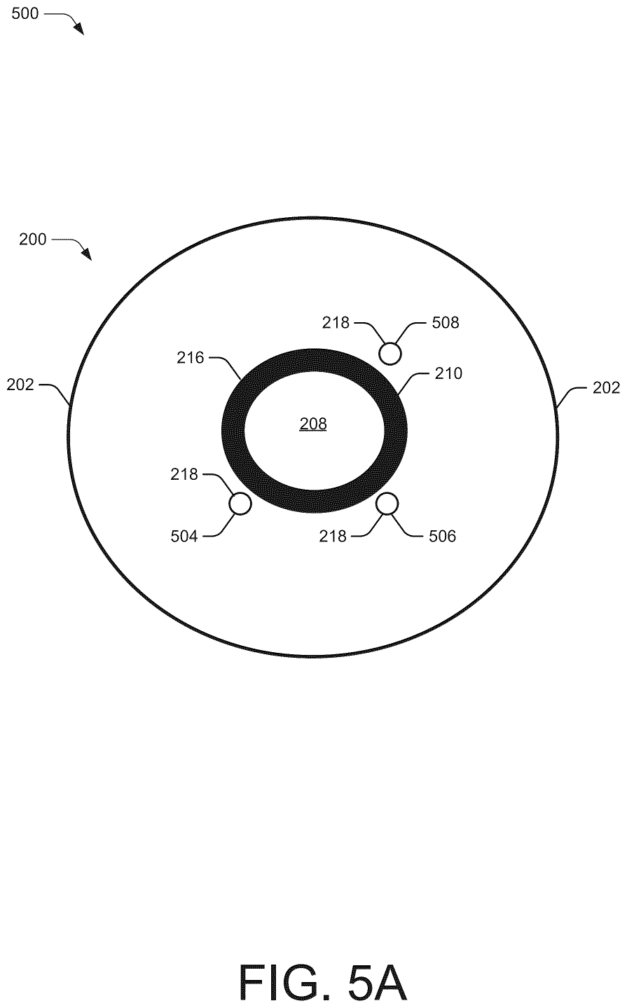

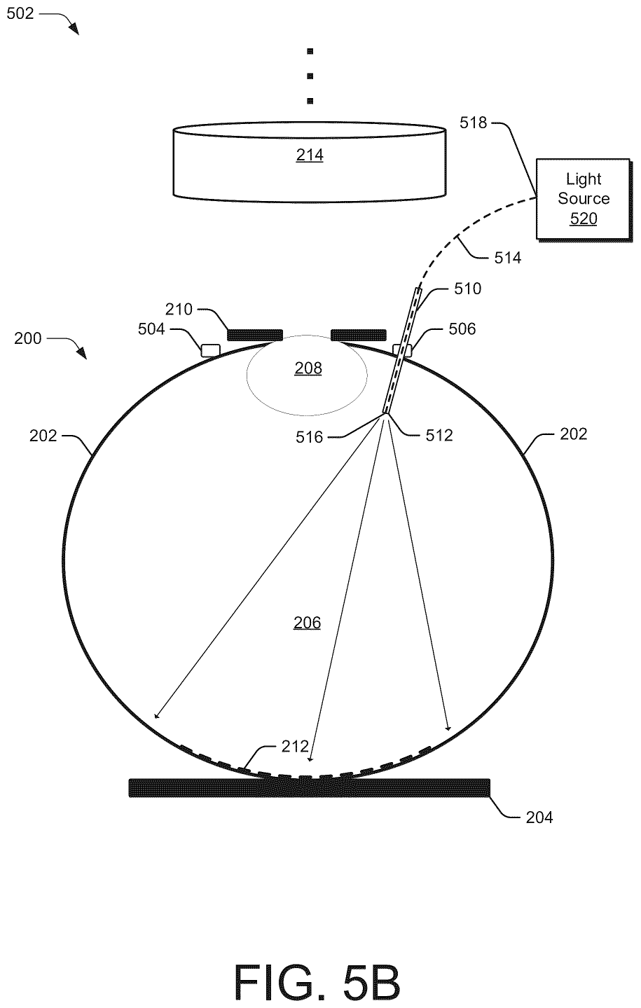

FIGS. 5A and 5B illustrate a tunable synthetic eye model 200 with an external light source, according to embodiments described herein. FIG. 5A illustrates a representation 500 of a top view of the eye model 200 with the external light source. FIG. 5B illustrates a representation 502 of a cross-sectional view of the eye model 200 with the external light source. With reference to FIG. 5A, in some embodiments, the eye model 200 includes cannulas 504, 506, and 508 which are disposed in auxiliary orifices 218 of the housing 202 separate from the opening 216.

As shown in the representation 500, the cannulas 504, 506, and/or 508 are disposed in portions of the housing 202 that correspond to the pars plana 104 of the human eye where cannulas are inserted in order to perform vitreoretinal surgical procedures. Accordingly, the cannulas 504, 506, and/or 508 simulate cannulas used in a vitreoretinal surgical procedure. With reference to FIG. 5B, the representation 502 includes an endoilluminator 510 disposed in the cannula 506 such that a distal end 512 of the endoilluminator 510 is disposed in the inner chamber 206. In one or more embodiments, the tunable synthetic eye model 200 and the endoilluminator 510 are part of a system for evaluating performance characteristics of the ophthalmic microscope 214. In some examples, the system can include multiple endoilluminators 510, such as one in each of the cannulas 504, 506, and 508.

At least one optical fiber 514 is disposed within the endoilluminator 510, and a distal end 516 of the at least one optical fiber 514 is also disposed in the inner chamber 206. For instance, a proximal end 518 of the at least one optical fiber 514 is disposed in an external light source 520 which may be a light source of an ophthalmic surgical console or a standalone light source. Light from the light source 520 enters the proximal end 518 of the at least one optical fiber 514 and is transmitted out from the distal end 516 of the at least one optical fiber to illuminate the inner chamber 206 and the optical target 212. By illuminating the optical target 212 using the endoilluminator 510 in this manner, the optical target 212 can be visualized through the lens 208 using the ophthalmic microscope 214 under lighting conditions that simulate lighting conditions of a vitreoretinal surgical procedure. In some embodiments, these simulated lighting conditions also comply with standards for optical radiation safety of medical devices for illuminating the interior of the human eye (e.g., the vitreous chamber 102) during a surgical procedure.

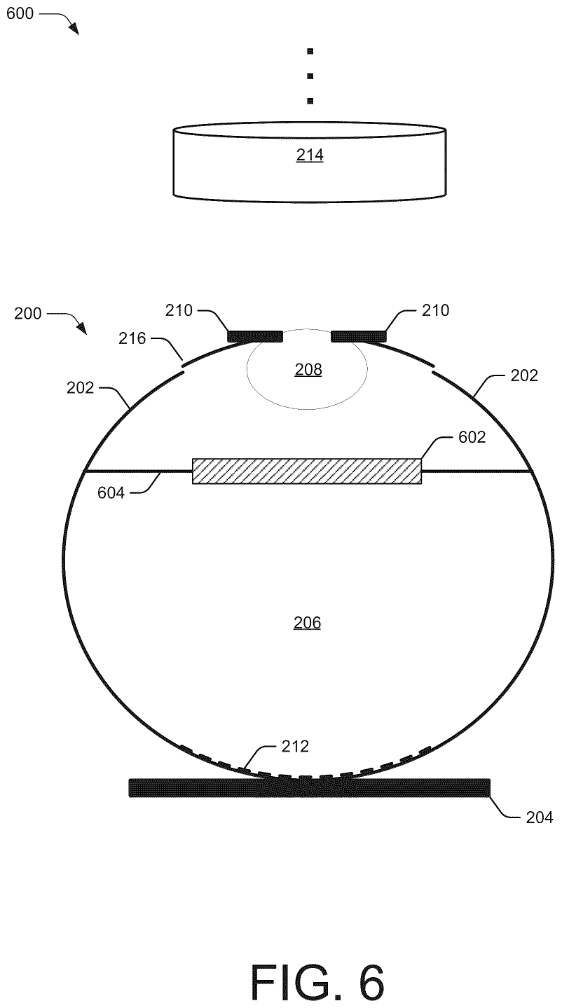

FIG. 6 illustrates a representation 600 of a cross-sectional view of a tunable synthetic eye model 200 with a tunable optical element, according to embodiments described herein. As shown, the representation 600 includes a tunable optical element 602 disposed within the inner chamber 206. For example, the tunable optical element 602 is oriented within the inner chamber 206 by a substrate 604.

In one or more embodiments, the tunable optical element 602 includes a liquid crystal (in addition or alternative to an example in which the lens 208 is a liquid lens) which is configured to provide a tunable or variable opaque effect (e.g., to simulate a cataract effect for the lens 208). In some examples in which the tunable optical element 602 includes a liquid crystal, the substrate 604 may apply an electric field to the liquid crystal which can change the opaque effect by increasing or decreasing a transparency of the tunable optical element 602. In such examples, changing the opaque effect can vary a simulated cataract effect for the lens 208, for example, from an effect that simulates a healthy natural lens 110 to an effect that simulates a cataractous lens 110 with varying degrees of opacity for different types or classes of cataracts. In certain embodiments, the tunable optical element 602 may include layers of liquid crystal, and an opacity of the tunable optical element 602 can be changed in substantially real time by changing polarizations of the layers of liquid crystal.

In some examples, the tunable optical element 602 is configured to generate a visual cataract effect for the lens 208 corresponding to opacities in a cataractous lens. For nuclear opacity grades ranging from standard 1 to standard 7, a “moderate” nuclear cataract is defined as having an opacity grade of 4.0 or greater. In various embodiments, the tunable optical element 602 generates the visual cataract effect for the lens 208 by increasing an opacity of the tunable optical element 602 to correspond to the opacity grade of 4.0 or greater.

In some embodiments, the tunable optical element 602 includes one or more filters such as a color filter, an interference filter, a polarizing filter, etc. For example, the tunable optical element 602 can include one or more lenses such as a focus tunable lens with a variable focal length (e.g., a liquid lens). In various embodiments, the tunable optical element 602 can be configured to simulate various conditions of the lens capsule such as a capsular cataract, posterior capsular opacification, pseudoexfoliation syndrome, and/or the like. In one or more embodiments, the tunable optical element 602 may be configured to simulate various conditions of the cornea 108 including, but not limited to, keratitis, corneal ectasia, corneal dystrophies, etc.

FIG. 7 illustrates a representation 700 of example optical targets, according to embodiments described herein. As depicted in FIG. 7, the representation 700 includes non-limiting examples 702-706. In example 702, the optical target 212 includes a 1951 United States Air Force (USAF) resolution test chart. In some embodiments, the optical target 212 of example 702 depicts groups of lines or bars at various orientations, and different groups include lines or bars with different widths and spacing between the lines or bars.

For example, a characteristic of the ophthalmic microscope 214 may be evaluated by visualizing (or capturing an image of) the optical target 212 of example 702 through the lens 208, and then determining a number of the lines or bars of the groups that can be visually distinguished and/or identifying a group having the smallest lines or bars that can be visually distinguished. In this example, the characteristic of the ophthalmic microscope 214 can include the modulation transfer function, the resolution limit, geometric distortions, etc. Further, the characteristic of the ophthalmic microscope 214 can also be evaluated using the optical target 212 of example 702 relative to the alternative lenses 302, 304, the light source 402, the endoilluminator 510, and/or the tunable optical element 602.

In example 704, the optical target 212 includes a Siemens star (e.g., a Siemens star test pattern, a Siemens star chart, etc.). In various embodiments, the optical target 212 of example 704 depicts equally-spaced radial lines extending outward from a central point. For instance, the radial lines depicted by the optical target 212 of example 704 generally form a circular pattern with high contrast between the radial lines and adjacent spacing between the radial lines. Widths of the radial lines and the adjacent spacing decrease from the outer circumference of the circular pattern towards the central point such that the radial lines become visually indistinguishable at the central point.

In some examples, the characteristic of the ophthalmic microscope 214 can be evaluated by visualizing (or capturing an image of) the optical target 212 of example 704 through the lens 208, and then identifying the closest radial lines to the central point that are visually distinguishable. In these examples, the closest radial lines to the central point that are visually distinguishable may indicate the resolution of the ophthalmic microscope 214. In addition to the resolution, the characteristic of the ophthalmic microscope 214 can also include the modulation transfer function, focus depth, field depth, distortion, and/or the like. In a manner similar to example 702, the characteristic of the ophthalmic microscope 214 can be evaluated using the optical target 212 of example 704 with the alternative lenses 302, 304, the light source 402, the endoilluminator 510, and/or the tunable optical element 602.

In example 706, the optical target 212 includes a field of view target. In one or more embodiments, the optical target 212 of example 706 depicts concentric circles separated by a particular distance. In various examples, the optical target 212 of example 706 may be used for calibrating the ophthalmic microscope 214. For example, by visualizing the optical target 212 of example 706 using the ophthalmic microscope 214 at a specific magnification and counting the number of visible concentric circles, the particular distance can be used to compute a calibration factor for the ophthalmic microscope 214. In some examples, the characteristic of the ophthalmic microscope 214 can be evaluated using the optical target 212 of example 706, the calibration factor, known dimensions of calibration objects which may be visualized using the ophthalmic microscope 214, the alternative lenses 302, 304, the light source 402, the endoilluminator 510, the tunable optical element 602, etc.

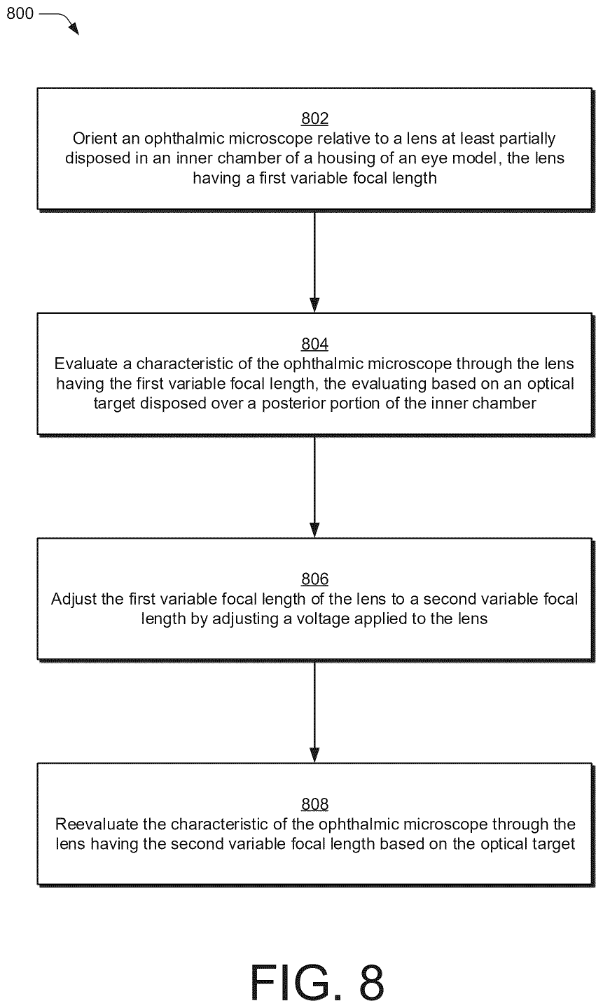

FIG. 8 illustrates a method 800 for evaluating a characteristic of an ophthalmic microscope. At 802, an ophthalmic microscope is oriented relative to a lens at least partially disposed in an inner chamber of a housing of an eye model, the lens having a first variable focal length. In various embodiments, the ophthalmic microscope 214 may be oriented relative to the lens 208, which is at least partially disposed in the inner chamber 206 of the housing 202. In some examples, the lens 208 has the first variable focal length based on a voltage applied to a portion of the lens 208 by the focal length controller 208C.

At 804, a characteristic of the ophthalmic microscope is evaluated through the lens having the first variable focal length, the evaluating based on an optical target that is disposed over a posterior portion of the inner chamber. In some embodiments, the characteristic of the ophthalmic microscope 214 may be evaluated through the lens 208 based on the optical target 212 that is disposed over the posterior portion of the inner chamber 206. In one or more examples, the characteristic of the ophthalmic microscope 214 can be evaluated through the lens 208 having the first variable focal length based on the optical target 212 of example 702 by identifying a first group of lines or bars having the smallest lines or bars that can be visually distinguished.

At 806, the first variable focal length of the lens is adjusted to a second variable focal length by adjusting a voltage applied to the lens. In various embodiments, the first variable focal length of the lens 208 is adjusted to the second variable focal length by adjusting the voltage applied to the portion of the lens 208 by the focal length controller 208C. In some examples, the first variable focal length of the lens 208 may be changed (e.g., by user interaction with the focal length controller 208C) to evaluate the characteristic of the ophthalmic microscope 214 through the lens 208 having the second variable focal length.

At 808, the characteristic of the ophthalmic microscope is reevaluated through the lens having the second variable focal length based on the optical target. In one or more embodiments, the characteristic of the ophthalmic microscope 214 may be evaluated through the lens 208 having the second variable focal length based on the optical target 212 of example 702 by identifying a second group of lines or bars having the smallest lines or bars that can be visually distinguished. In some examples, a difference between the size of the lines or bars of the first group and the size of the lines or bars of the second group may correspond to the achievable resolution of the ophthalmic microscope 214 through the lens 208 having the first variable focal length and through the lens 208 having the second variable focal length.

It is to be appreciated that one or more parameters of the lens 208 and/or the eye model 200 can be modified in order to evaluate the same characteristic of the ophthalmic microscope 214 and/or a different characteristic of the ophthalmic microscope 214. In one or more examples, an amount of light illuminating the inner chamber 206 can be changed to evaluate one or more characteristics of the ophthalmic microscope 214 under different lighting conditions. In some embodiments, the lens 208 can be replaced by the alternative lenses 302, 304 to evaluate characteristics of the ophthalmic microscope 214. In certain embodiments, one or more characteristics the ophthalmic microscope 214 may be evaluated through the lens 208 having the first variable focal length and/or the second variable focal length based on a different optical target 212.

The above-disclosed subject matter is to be considered illustrative, and not restrictive, and the appended claims are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An eye model comprising:
a housing having an inner chamber and at least one opening adjacent to an anterior portion of the inner chamber;
a lens at least partially disposed in the at least one opening, the lens having a variable focal length; and
an optical target disposed over a posterior portion of the inner chamber, the optical target configured to facilitate evaluation of a characteristic of an ophthalmic microscope when viewed through the lens with the ophthalmic microscope.

2. The eye model of claim 1, further comprising an adjustable aperture configured to vary an amount of light passing through the lens into the inner chamber.

3. The eye model of claim 2, wherein the adjustable aperture is disposed over the lens.

4. The eye model of claim 1, wherein the variable focal length is adjustable by changing at least one of a thickness or a curvature of the lens.

5. The eye model of claim 1, wherein the lens comprises a liquid lens.

6. The eye model of claim 1, further comprising a tunable optical element configured to generate a visual cataract effect for the lens corresponding to opacities in a cataractous lens.

7. The eye model of claim 1, wherein the variable focal length is variable by adjusting a voltage applied to the lens.

8. The eye model of claim 1, wherein the housing is configured to house an endoilluminator for illuminating the inner chamber.

9. The eye model of claim 1, wherein the characteristic of the ophthalmic microscope includes at least one of modulation transfer function, distortion, field of view (FOV), depth of field (DOF), or relative illumination.

10. The eye model of claim 1, wherein the ophthalmic microscope comprises a fundus camera.

11. The eye model of claim 1, wherein the optical target includes at least one of a 1951 United States Air Force resolution test chart, a Siemens star, or a field of view target.

12. The eye model of claim 1, wherein the inner chamber comprises a balanced salt solution (BSS) or a simulated vitreous humor gel.

13. A device comprising:
a housing comprising an inner chamber;
a first interchangeable lens of a set of interchangeable lenses for evaluating one or more characteristics of an ophthalmic microscope, the first interchangeable lens at least partially disposed in the housing; and
a curved optical target disposed over a portion of the inner chamber, the curved optical target configured to facilitate evaluation of a first characteristic of the ophthalmic microscope when viewed through the first interchangeable lens with the ophthalmic microscope.

14. The device of claim 13, wherein the first characteristic of the ophthalmic microscope includes at least one of modulation transfer function, distortion, field of view (FOV), depth of field (DOF), or relative illumination.

15. The device of claim 13, wherein the curved optical target is configured to facilitate evaluation of a second characteristic of the ophthalmic microscope when viewed through a second interchangeable lens of the set of interchangeable lenses.

16. The device of claim 13, further comprising a light source configured to illuminate the inner chamber.

17. The device of claim 16, wherein the light source comprises an endoilluminator.

18. A method comprising:
orienting an ophthalmic microscope relative to a lens at least partially disposed in an inner chamber of a housing of an eye model, the lens having a first variable focal length; and
evaluating a characteristic of the ophthalmic microscope through the lens having the first variable focal length, the evaluating based on an optical target disposed over a posterior portion of the inner chamber.

19. The method of claim 18, further comprising:
adjusting the first variable focal length of the lens to a second variable focal length by adjusting a voltage applied to the lens; and
reevaluating the characteristic of the ophthalmic microscope through the lens having the second variable focal length based on the optical target.

20. The method of claim 18, further comprising:
replacing the lens with an alternative lens; and
evaluating an additional characteristic of the ophthalmic microscope using the alternative lens.

21. The method of claim 18, further comprising:
replacing the lens with an alternative lens;
reevaluating the characteristic of the ophthalmic microscope with the alternative lens; and
comparing the characteristic of the ophthalmic microscope when using the lens and the characteristic of the ophthalmic microscope when using the alternative lens.
